(19) United States
(12) Patent Application Publication
(10) Pub. No.: US 2021/0260240 A1
(43) Pub. Date: Aug. 26, 2021

(54) BENDABLE SPLINT AND MOLDING METHOD THEREOF

(71) Applicant: Hong Sub YOON, Seoul (KR)

(72) Inventor: Hong Sub YOON, Seoul (KR)

(21) Appl. No.: 17/254,274

(22) PCT Filed: Jun. 19, 2019
(86) PCT No.: PCT/KR2019/007377

§ 371 Date: Dec. 19, 2020

(30) Foreign Application Priority Data
Jun. 19, 2018 (KR) 10-2018-0070367
Jun. 19, 2018 (KR) 10-2018-0112135

(51) Int. Cl. A61L 15/14; A61F 5/058; A61L 15/12; C08L 67/04

(52) U.S. Cl. CPC A61L 15/14; A61F 5/058; B29B 9/06; C08L 67/04; A61L 15/125

(57) ABSTRACT

The present invention relates to a splint bendable in a curved shape and a method of forming thereof. According to an embodiment of the present invention, there may be provided a splint bendable in a curved shape comprising a lower splint portion having a plurality of first ventilation holes formed at a certain interval; an extension splint portion in which a plurality of extension holes are formed at a certain interval; and an upper splint portion in which a plurality of second ventilation holes are formed at a certain interval, and wherein the lower splint portion, the extension splint portion, and the upper splint portion are bent into an arc shape by a first bending, and a second bending wherein the lower splint portion and the upper splint portion are bent at different angles by a plurality of extension holes is performed for the extension splint portion.

### CROSS-REFERENCES TO RELATED APPLICATION

The present application claims the benefit of Korean application No. 10-2018-0070367 filed on Jun. 19, 2018 and Korean application No. 2018-0112135 filed on Sep. 19, 2018, both of which are herein incorporated by reference in its entirety.

### BACKGROUND OF THE INVENTION

### 1. Field

The present invention relates to a splint and a molding method thereof, and more particularly, to a splint bendable with curvature and forming method thereof.

### 2. Description of the Related Art

In general, the splints may be used to protect fractured bone, injured muscle tissues and elongated or ruptured ligament by partially or entirely tightly winding an injured portion of a patient's body. Conventionally, method of winding and fixing a plaster-coated bandage on the injured portion has been widely used. However, this treatment procedure cannot effectively support the insured portion because a contraction of the plaster occurs during the curing of the plaster, and as there is no ventilation, itching may be caused when used for a long time. Since a separating cutter must be used to remove the plaster-coated bandage after the treatment is completed, it may cause a wound on the skin during removing the paster-coated bandage.

In order to solve the problem of the splint using the plaster-coated bandage, elastic fibers impregnated with a curable resin such as moisture-curable polyurethane in a polyester knitted fabric, a glass fiber knitted fabric, or a nonwoven fabric have recently been used. The elastic fiber impregnated with the curable resin is widely used with a splint with improved moldability by peeling off the wrapping paper during use so that the elastic fibers inside may be quickly naturally cured at room temperature.

The conventional splint as described above is mainly used because of its excellent moldability and curability during the treatment, but a cutter rotating at high speed is used on the surface of the elastic fiber to remove the splint after the injured body is cured. When the cured elastic fiber is cut by the cutter which rotates at high speed, dust particles are generated or high heat is generated in the process of cutting the elastic fiber by the cutter rotating at high speed. Therefore, there are serious accidents such as burns to the skin or injury of the skin attacked with elastic fibers.

Recently, in order to solve the above problems, an incision portion having a thickness thinner than the common thickness of the splint may be provided on one side portion of the splint in a longitudinal direction of the splint, and when removing after the patent's are cured, the incision portion having the thinner thickness may be cut with scissors or a knife to remove the splint from the patient. However, in this case, even though the fundamental purpose of the incision portion is to easily cut the splint, since the curable resin is uniformly coated or cured even on the incision portion as a whole, it is ever difficult to cut the incision portion with a cutting tool such as scissors or a knife, and therefore, it requires ever much efforts and strength. Thus, the efficiency of the treatment may be severely reduced. In addition, in the process for removing the splint from the injured body after the patient is cured, it may cause many problems such as damage to the skin.

In addition, since the conventional splint is formed by injection-molding method, each mold for the injection-molding method must be provided according to the body portions, for example, arm or leg of the patient who wears the splint. This may cause an increase in the mold cost since diverse moldings depending on the body portions are required.

In addition, in general, synthetic plastics are indispensable for modern life due to their excellent mechanical properties, low price, and low weight, and are used for various purposes all over the world. However, the synthetic plastics are not easily decomposed, and as a disadvantage, the synthetic plastics may cause environmental pollution problems more and more seriously. Specially, these synthetic plastics have a problem of causing the serious environmental pollution when they are disposed.

Accordingly, a technology to replace the synthetic plastics with biodegradable resins that can decompose in the natural state is being developed. The biodegradable resins are those that are naturally degraded by microorganisms in the soil. For example, polylactic acid (PLA), polyglycolic acid (PGA), poly caprolactone (PCL), aliphatic polyester resins, polyhydroxy butyric acid (PHBA), D-3-hydroxy butyric acid (HBA), and the like may be enumerated. However, the biodegradable resins, i.e. such as polylactic acid (PLA), polyglycolic acid (PGA), polycaprolactone (PCL), aliphatic polyester resins, polyhydroxy butyric acid (PHBA) and D-3-hydroxy as described above have poor mechanical properties, for example, in terms of dimensional stability, tensile strength and hardness.

In order to reinforce such physical properties of the biodegradable resins, methods of manufacturing the biodegradable resin by laminating additionally a reinforcing sheet to a biodegradable resin sheet or by adding other general resins or inorganic fillers to the biodegradable resin during extrusion molding of the biodegradable sheet have been attempted. However, a sheet using a conventional biodegradable resin has to be processed at a high temperature in order to mold the biodegradable resin to have a desired shape, so processability of the biodegradable resin is deteriorated, and there is a problematic defect that the biodegradable resin cannot meet standards in relation with bio-contact and food-contact.

### SUMMARY OF THE INVENTION

The technological object to be achieved by the present invention is to provide a splint bendable in a curved shape that may freely form the shape, size, bending angle of the splint according to the portion of the patient body.

The technological object to be achieved by the present invention is to provide a biodegradable composite resin composition using an eco-friendly manufacturing process and improved product usability.

In addition, the technological object to be achieved by the present invention is to provide a method for manufacturing a biodegradable composite resin with enhanced low-temperature processability, which is harmless to bio-contact and improved biodegradability, enabling resource recycling, and a method for manufacturing a sheet manufactured therefrom.

A splint bendable in a curved shape according to an embodiment of the present invention for solving the above problem includes: a lower splint having a plurality of first ventilation holes formed at regular intervals; an extension splint portion in which a plurality of extension holes are formed at regular intervals; and an upper splint portion in which a plurality of second ventilation holes are formed at regular intervals. The lower splint portion, the extension splint portion, and the upper splint portion are bent into an arc shape by first bending, and the extension splint portion performs a second bending for the lower splint portion and the upper splint portion at different angles by a plurality of extension holes.

The extension splint portion is characterized in that a plurality of extension holes are arranged at regular intervals so that the lower splint portion and the upper splint portion may be bent smoothly by second bending.

In one embodiment, the extension splint portion is characterized in that it includes the first extension hole extending which is formed in a predetermined length in the middle of the extension splint portion, and is extended vertically apart by second bending; and the second extension hole which is formed on both sides of the first extension hole in a predetermined length, respectively, and extended vertically apart by second bending.

In addition, a splint bendable in a curved shape according to an embodiment of the present invention is characterized in that it comprises a lower splint portion having a plurality of first ventilation holes, second ventilation holes, and third ventilation holes formed at regular intervals; an upper splint portion extending on an upper side of the lower splint portion; an extension splint portion extending to one side of the upper splint portion and having a plurality of extension holes formed at regular intervals; and the lower splint portion and the upper splint portion are bent into an arc shape by first bending, and a second bending is applied to the extension splint portion so that it may be bent to have a predetermined diameter by a plurality of extension holes.

In addition, the method for molding a splint bendable in a curved shape is characterized in that it includes a step for cutting a splint member into a predetermined shape according to the treatment portion of the splint; a step for forming a plurality of extension holes in the extension splint via a perforation process to smoothly transform and extend the splint member when the splint member is bent; a step for forming a plurality of ventilation holes via a perforation process to allow air to communicate with the outside according to the treatment portion of the splint member; a first bending step for bending the splint member into a predetermined arc shape so as to surround the outer surface of the treatment portion of the splint member; and a second bending step for bending the extension splint portion having the extension hole formed thereon to have a different angle or a predetermined diameter.

The biodegradable composite resin composition with enhanced low-temperature processability according to an embodiment of the present invention for solving the above other problems is formed by mixing PLA (Poly lactic acid), PCL (polycaprolactone), PBS (polybutylene succinate), PBAT (polybutylene adipate-co-terephthalate), PVAC (polyvinyl acetate), a cross-linking agent, and a compatibilizer.

In an embodiment, in connection with the biodegradable composite resin composition with enhanced low-temperature processability, 28 to 72% by weight of PLA Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, 1 to 3% by weight of compatibilizer may be melt-extruded through an extruder.

The PLA may be a stereo-complex of PLDA which is an isomer of PLLA, and the PLA may be a homopolymer such as poly-L-lactide, poly-D-lactide and poly-DL-lactide, or PLA may be a copolymer including poly-L-lactide, poly-D-lactide and poly-DL-lactide.]

In one embodiment, in the case of a copolymer including the poly-L-lactide, poly-D-lactide, and the poly-DL-lactide, 5 to 10% by weight of the poly-D-lactide may be a stereo complex.

In addition, the PVAC (polyvinyl acetate) may be selected from the group consisting of PVOH (polyvinyl alcohol) linked with a cross-linking agent and derivatives or mixtures thereof.

In one embodiment, the PBS (polybutylene succinate) may be replaced with PBSA (polybutylene succinate adipate), and the compatibilizer may be MAH (maleic anhydride).

A method for manufacturing a biodegradable composite resin according to an embodiment of the present invention for solving the above other problems comprises a step S110 for powdery processing 28 to 72% by weight of PLA Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, 1 to 3% by weight of compatibilizer; a step S120 for mixing the powdery processed raw material by using a double blade ribbon blender; a step S130 for performing melting-extrusion of the mixed raw material using a twin extruder equipped with a raw material supply device; a step S140 for injecting the melt-extruded raw material into a die, and then cooling and drying the strands outputted through the die; and a step S150 for pelletizing the cooled strand through a cutting machine and packaging it.

In one embodiment, the strand has the specification defining density of 1.25±0.05 (g/cm²), tensile strength of 50 (Mpa), tensile activity rate of 3.5˜6 (Gpa), softening temperature of 60˜70° C., shrinkage less than 0.5% and moisture content less than 200 ppm.

In the step S140 for cooling and drying the melt-extruded strand, the processed strand may be cooled with an air cooling system.

The sheet manufacturing method comprises; a step S210 for powder processing 28 to 72% by weight of PLA (Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, and 1 to 3% by weight of a compatibilizer; a step S220 for mixing the raw material processed into a powder state with a double blade ribbon blender; a step S230 for melting-extrusion of the mixed raw material using a twin extruder equipped with a raw material supply device; a step S240 for injecting the melt-extruded raw material into a die and then cooling and drying the strands outputted through the die); a step for S250 for melting and extruding the cooled and dried strand using a T-die extruder for sheet manufacturing; a step S260 for manufacturing a sheet by rolling the melt-extruded composite resin through the T-die extruder to control the thickness and to perform a primary cooling; a step for S270 for applying a secondary cooling to the sheet cooled by the primary cooling in a state in which the thickness is adjusted; and a step S280 for cutting and packaging the sheet cooled by the secondary cooling according to standards.

In one embodiment, during thickness adjustment via a rolling and the primary cooling step S260, a three-axis roller including a cooling roll may be applied.

According to an embodiment of the present invention, there may be provided a splint bendable in a curved shape, which may be molded according to the treatment or wearing portion of the splint, and wherein the splint member may be freely molded according to the body portion to be splint, such as a leg or arm, and when the splint member is bent, it is configured that an extension hole in the extension splint portion corresponding to the transformation of the splint member, thereby facilitating transformation due to bending, and preventing damage or destruction of the splint member due to transformation,

In addition, according to an embodiment of the present invention, a method for molding a splint bendable in a curved shape may be provided, wherein the splint member may be freely molded into a desired shape and size by first bending and second bending, there is no need for a mold required for forming the splint and thus, manufacturing cost according to the molding, and a molding may be performed by adjusting the strength of the splint according to the spacing, size, and length of the extension splint portion.

According to another embodiment of the present invention, there may be provided a biodegradable composite resin composition with enhanced low-temperature processability wherein a product manufactured to have a predetermined shape may be easily post-processed at a low temperature of 60 to 70° C. and thus, convenience of use is improved, is harmless to bio-contact and is capable of producing a sheet conforming to food contact standards, and a method of manufacturing the same.

In addition, a method of manufacturing a biodegradable composite sheet that satisfies the biodegradability condition and enables recycling of resources by using the above manufacturing method may be provided.

### BRIEF DESCRIPTION OF THE DRAWINGS

FIG. 1 is a diagram illustrating a splint bendable in a curved shape according to an embodiment of the present invention.

FIG. 2 is a three-dimensional diagram illustrating a shape of a splint bendable in a curved shape observed from one side according to an embodiment of the present invention.

FIG. 3 is a three-dimensional diagram illustrating a shape of a splint bendable in a curved shape observed from the other side according to an embodiment of the present invention.

FIG. 4 is a diagram illustrating a splint bendable in a curved shape according to another embodiment of the present invention.

FIG. 5 is a three-dimensional diagram illustrating a splint bendable in a curved shape according to another embodiment of the present invention.

FIG. 6 is a flow chart illustrating a method of forming a splint bendable in a curved shape according to an embodiment of the present invention.

FIG. 7 is a flow chart illustrating a method of manufacturing a biodegradable composite resin with enhanced low-temperature processability according to an embodiment of the present invention

FIG. 8 is a flow chart illustrating a method of manufacturing a sheet using a biodegradable composite resin with enhanced low-temperature processability according to an embodiment of the present invention.

### DETAILED DESCRIPTION OF THE INVENTION

Hereinafter, the preferred embodiments of the present invention will be described in detail with reference to the accompanying drawings.

The embodiments of the present invention are provided to more fully describe the present invention to those having a common knowledge in the related art, and the following embodiments may be modified in various other forms, and the scope of the present invention is not limited to the following embodiments. Rather, these embodiments are provided to explain the present invention more clearly and complete, and to fully convey the spirit of the present invention to those skilled in the art.

In addition, in the following drawings, the same reference numerals in the drawings refer to the same elements. As used herein, the term, “and/or” includes any one and all combinations of one or more of the listed items.

The terminology used herein is used to describe a specific embodiment and is not intended to limit the present invention. As used herein, a singular form may include plural forms unless the context clearly indicates otherwise. Also, as used herein, the term such as “comprise” and/or “comprising” specifies the mentioned shapes, numbers, steps, actions, members, elements and/or the presence of these groups, and does not exclude the presence or addition of one or more other shapes, numbers, actions, members, elements and/or presence or addition of groups.

Reference to a layer formed “on” a substrate or other layer herein refers to a layer formed directly on the substrate or other layer; or also may refer to an intermediate layer formed on the substrate or other layer, or a layer formed on intermediate layers. Further, for those skilled in the art, a structure or shape arranged “adjacent” to another shape may have a portion disposed below or overlapping the adjacent shape.

In this specification, as shown on the drawings, the relative terms such as “below”, “above”, “upper”, “lower”, “horizontal” or “vertical” may be used to describe the relationship between one component member, one layer, or one region and another component member, another layer, or another region. It is to be understood that these terms encompass not only the orientation indicated in the figures, but also other orientations of the device.

Hereinafter, the embodiments of the invention will be described with reference to cross-sectional diagrams schematically showing ideal embodiments (and intermediate structures) of the invention. In the drawings, for example, the size and shape of the members may be exaggerated for convenience and clarity of description, and in actual implementation, modifications of the illustrated shape may be expected. Accordingly, embodiments of the present invention should not be construed as limited to the specific shapes of the members or regions shown herein. In addition, reference numerals of members in the drawings refer to the same members throughout the drawings.

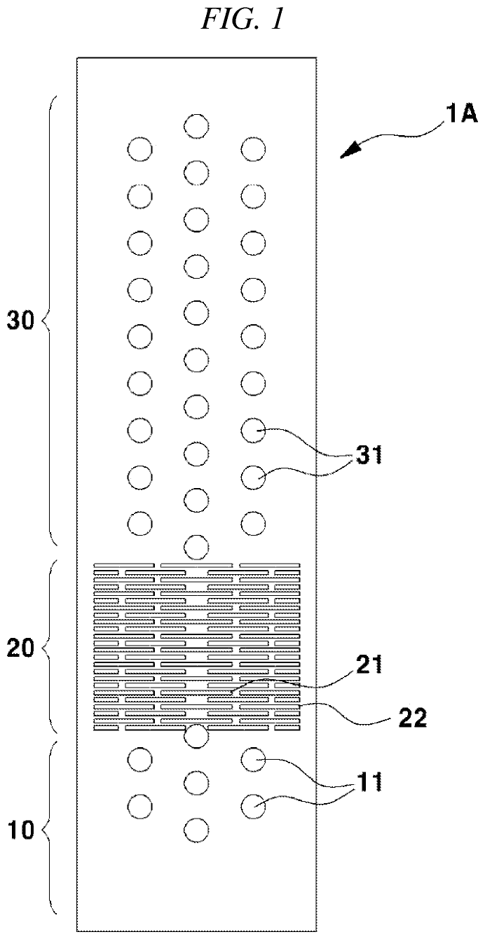

FIG. 1 is a diagram illustrating an initial shape of a splint bendable in a curved shape before forming the splint according to an embodiment of the present invention. The splint 1A before forming the splint 1A may be obtained by cutting a splint member into a predetermined shape and size depending on a site of a patient, or treatment area. The splint member may be made of a synthetic resin material having a certain thickness, and the splint member may be formed in various shapes according to the treatment site of the splint.

The splint 1A is a splint worn on a leg as an embodiment of the present invention, and may include a lower splint portion 10 supporting a foot, an extension splint portion 20 bendable to cover a heel of the foot, and an upper splint portion 30 in close contact with a calf of the leg.

The splint 1A before forming the splint 1A may be manufactured from a splint member of a synthetic resin plate having a predetermined length and width. The lower splint portion 10 is formed to have a predetermined width and length to support the foot, and the extension splint portion 20 may be integrally extended from the lower splint portion 10 to facilitate bending of the splint 1A when the splint is bent. In addition, the upper splint portion 30 may be formed to have the same width as the lower splint 10.

The lower splint portion 10 may include a plurality of first ventilation holes 11 to allow air to pass from the outside to the inside when the splint 1A is worn or applied to the patient. The first ventilation holes 11 may be formed in plural, and, if needed, the first ventilation holes 11 may be formed at predetermined intervals or the first ventilation holes 11 may have different diameters to each other. The first ventilation hole 11 may be formed in various shapes such as a triangle, a square, an oval type, a star shape, and a heart shape, as well as a circle.

An extension splint portion 20 may be provided on the upper side of the lower splint portion 10 to facilitate bending of the splint 1A during the second bending. In the extension splint portion 20, a plurality of extension holes 21 and 22 may be formed at regular intervals to facilitate bending of the splint 1A by secondarily bending the extension splint portion 20.

The extension splint portion 20 may include a first extension hole 21 formed to have a predetermined length in the middle of the extension splint portion 20 and extending vertically apart by the second bending of the extension splint portion 20, and second extension hole 22 formed at both sides of the first extension hole 21 to have a predetermined length and extended vertically apart by the second bending.

When the lower splint portion 20 and the upper splint portion 30 are bent at different angles, the extension splint portion 20 may be extended and freely transformed so that the extension splint portion 20 may be made into a desired shape according to the shape and size of the splint 1A.

In the extension splint portion 20, a first extension hole 21 formed to have a predetermined width, and a second extension hole 21 having the same width and length as the first extension hole 21 on both sides of the first extension hole 21 may be provided, respectively. The first extension hole 21 and the second extension hole 22 may be formed in the width direction of the splint 1A, and a plurality of the first extension hole 21 and the second extension hole 22 may be formed at regular height intervals.

An upper splint portion 30 may be integrally formed on the upper side of the extension splint portion 20. A plurality of second ventilation holes 31 may be formed in the upper splint portion 30 at regular intervals to allow air to pass through from the outside. The second ventilation hole 31 may be formed in various shapes such as a triangle, a square, an oval type, a star shape, and a heart shape, as well as a circle.

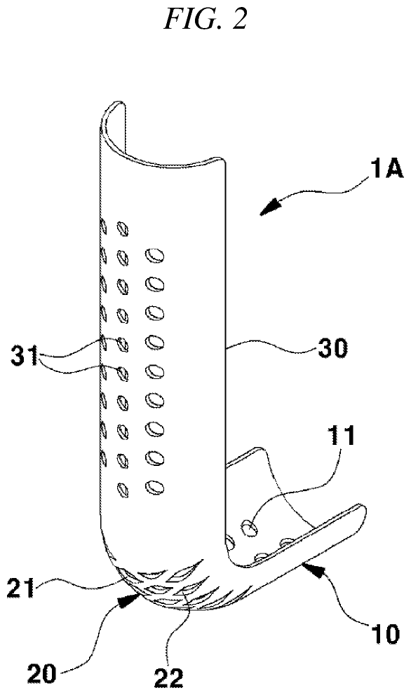

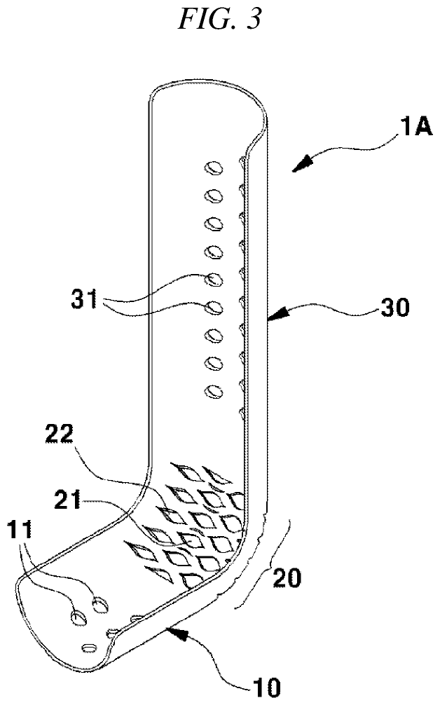

FIG. 2 is a three-dimensional diagram illustrating a shape observed from one side after forming a splint bendable in a curved shape according to an embodiment of the present invention, and FIG. 3 is a three-dimensional diagram showing a shape observed from the other side after forming a splint bendable in a curved shape according to an embodiment of the present invention.

The splint 1A formed after forming of the splint 1A, which are shown in FIGS. 2 and 3 is formed according to the shape of a leg, which is a treatment site. Referring FIG. 2 and FIG. 3, the splint 1A formed after forming of the splint 1A may include a lower splint portion 10 in which the first ventilation hole 11 is formed, an extension splint portion 20 extending from the lower splint portion 10, and an upper splint portion 30 disposed at an angle different from the lower splint portion 10.

In connection with the lower splint 10, the extension splint portion 20, the upper splint portion 30, the first ventilation hole 11, the first extension hole 21, the second extension hole 22, the second ventilation hole 31, a description of the corresponding configuration of FIG. 1 may be referred to.

As for the splint 1A bendable in a curved shape, the splint member is cut into an appropriate size and shape according to the shape of the area of a patient to be treated, and then, first of all, the splint member may be cut and then be firstly bent into a predetermined arc or diameter, and then secondarily bent depending on the treatment site of the patient. Accordingly, by forming the splint 1A having an appropriate shape and size by the first and second bending of the splint 1A according to the treatment area of the patient, the splint 1A may be formed in an appropriate size and shape for the portion of the patient's body, and the patient may wear comfortably the splint 1A without the feeling of pressure or tightening caused by wearing the splint 1A.

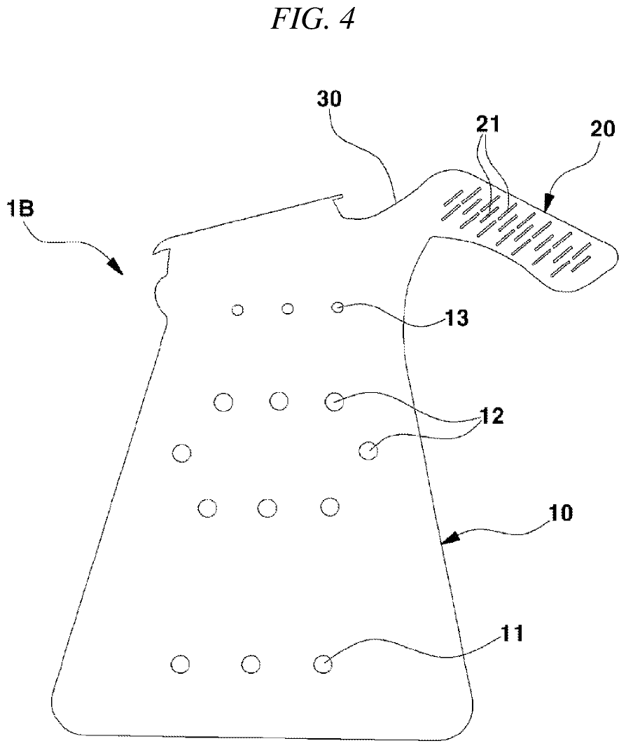

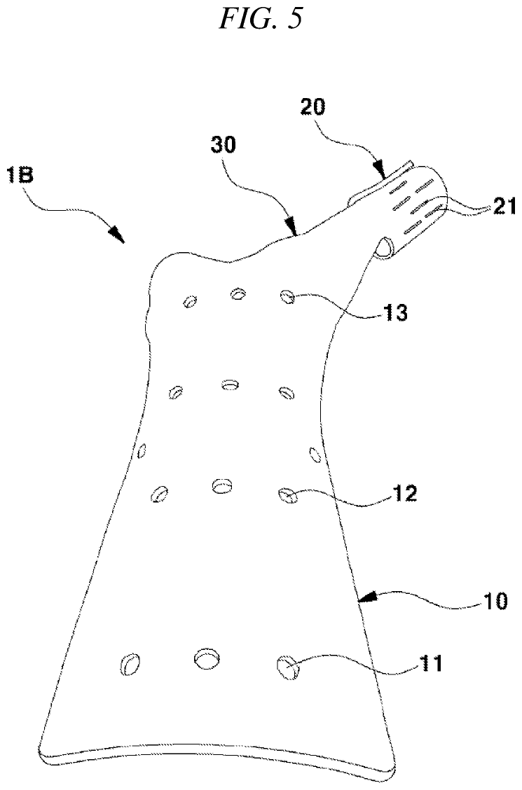

FIG. 4 is a diagram illustrating a shape of a splint bendable in a curved shape before forming the splint according to another embodiment of the present invention, and FIG. 5 is a three-dimensional diagram illustrating a shape of a splint bendable in a curved shape after forming the splint according to another embodiment of the present invention.

The splint 1B represents a splint for wearing on an arm or treating an arm as an embodiment of the present invention, and the splint 1B represents a splint before forming the splint 1B, and the splint 1B shown in FIG. 5 shows a formed splint so that the splint member shown in FIG. 4 may be applied to the arm.

In one embodiment, the splint 1B includes a lower splint portion 10 where a plurality of first ventilation holes 11, second ventilation holes 12 and third ventilation holes 13 are formed at regular intervals; an upper splint portion 30 extending on one side of an upper portion of the lower splint portion 10; and an extension splint portion 20 extending to one side of the upper splint portion 30, and having a plurality of extension holes 21 formed at regular intervals. The lower splint portion 10 and the upper splint portion 30 may be bent into an arc shape by a first bending, and a second bending may be applied to the extension splint portion 20. The extension splint portion 20 may be bent into a shape having a predetermined diameter by a plurality of extension holes.

Referring to FIG. 4, in the splint 1B before forming, the lower splint portion 10 and the upper splint portion 30 may be integrally formed, and an extension splint portion 20 may be integrally formed on one side of the upper splint portion 30. In addition, the lower splint portion 10 and the upper splint portion 30 may be integrally formed, and the extension splint portion 20 may be formed to extend to one direction of an upper side of the upper splint portion 30.

In the lower splint 10, a plurality of first ventilation holes 11, second ventilation holes 12 and third ventilation holes 13 may be formed to allow air to pass from the outside to the inside of the splint 1B during the wearing of the splint 1B. The upper splint portion 30 may be formed on an upper side of one side of the lower splint portion 10 to have a predetermined length.

The extension splint portion 20 may be formed to extend from the upper splint portion 30, and the extension splint portion 20 may be extended to a predetermined length so that a finger may be inserted and splinted. In the extension splint portion 20, a plurality of extension holes 21 may be formed at regular intervals to facilitate transformation caused by the second bending.

Referring to FIG. 5, a splint 1B bendable in a curved shape is used to splint the arm, wrist, and fingers, and the lower splint portion 10 is formed as a shape surrounding the arm, and the extension splint portion 20 may be formed to be able to splint a finger or an arm by inserting the finger.

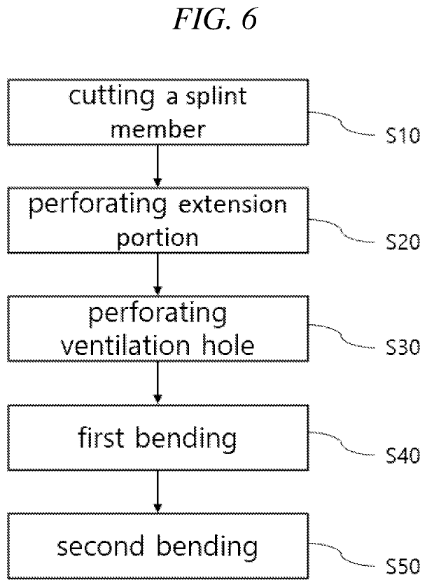

FIG. 6 is a flow chart illustrating a method of forming a splint bendable in a curved shape according to an embodiment of the present invention.

A method of forming a splint bendable in a curved shape according to an embodiment of the present invention comprises; a step S10 for cutting the splint member into a predetermined shape according to the treatment portion of the splint; a step S20 for forming a plurality of extension holes in an extension splint portion through a perforating process to facilitate deformation and extension of the splint member when the splint member is bent; a step S30 for forming a plurality of ventilation holes through a perforating process to allow air to communicate with the outside according to a treatment portion of the patient; a first bending step S40 for bending the splint member into a predetermined arc shape so as to surround an outer surface of the treatment portion of the patient; and a second bending step S50 for performing a curved bending the extension splint portion on which the extension hole is formed to have a different angle or a predetermined diameter.

In one embodiment, the method of forming a splint bendable in a curved shape comprises cutting the splint member into a predetermined shape according to the splint applied to the leg or arm, as shown in FIGS. 1 to 6 (S10).

The splint member may be cut into a substantially rectangular shape having a predetermined length and width, as shown in FIG. 1 when the splint member is a splint that is operated or worn on a leg. In addition, in the case of a splint that is applied or worn on an arm, as shown in FIG. 4, a lower splint portion 10 having a predetermined length and width is formed into an approximately trapezoidal shape, and an extension splint portion 20 is formed integrally, so that a finger may be inserted. That is, the splint member may be formed into an appropriate shape according to a portion of the body to be worn such as a leg or an arm, and one or more extension splint portions 20 may be formed to be worn by inserting a portion of the body such as a finger or a toe.

In the splint member, extension holes 21 and 22 having a predetermined length and width may be formed at a portion where transformation occurs due to bending (S20). The extension hole 20 not only facilitates the transformation of the splint member through a bending process, but also allows the splint member to be transformed into a desired shape when the splint member is transformed. In other words, the extension holes 21 and 22 are formed to have a straight line so that the gaps between the extension holes 21 and 22 may be separated from each other when being transformed by the bending, and therefore the bending may be performed smoothly and excellently, and the splint 1B may be bent into a desired shape despite the stress caused by the bending.

In addition, in the splint member, a plurality of ventilation holes 11, 12, 13 may be formed to allow air to pass from the outside to the inside of the splint when the splint is worn or treated (S30). The ventilation holes 11, 12, 13 may be formed in order to provide contact with external air to such as arms or legs according to the wearing of the splint, so as to relieve the feeling of heaviness and discomfort caused by the wearing of the splint. In this way, in a state in which the extension splint portion 20 and the ventilation holes 11, 12, 13 are formed in the splint member, first bending may be performed by a bending machine (S40).

In this first bending, as shown in FIGS. 2 and 3, the lower splint portion 10 and the upper splint portion 30 may be bent into a predetermined arc shape. That is, the splint member may be bent into an approximately semicircular shape by the first bending. In addition, the first bending is to bend the lower splint portion 10 into a predetermined arc shape, as shown in FIG. 5. That is, the splint member may bend the lower splint portion 10 into a predetermined arc shape by the first bending as shown in FIG. 5.

The second bending may be performed for the splint member which is subject to the first bending may be so that the lower splint portion 10 and the upper splint portion 30 may be bent at different angles based on the extension splint portion 20 (S50). As shown in FIG. 2 and FIG. 3, the splint worn on the leg may be bent at a right angle or may be bent at a desired angle by a bending machine. In addition, as shown in FIG. 5, the splint worn on the arm may bend the extension splint portion 20 into a circular shape having a predetermined diameter in a state in which the first bending is performed.

According to an embodiment of the present invention, it is possible to provide a biodegradable composite resin composition with enhanced low-temperature processability, a method of manufacturing a composite resin, and a method for manufacturing a sheet therefrom. It may be used as a splint member for manufacturing a splint bendable in a curved shape using a biodegradable composite resin composition or sheet to be described later.

According to an embodiment of the present invention, a biodegradable composite resin composition having enhanced low-temperature processability which is characterized in that PLA (Poly lactic acid), PCL (polycaprolactone), PBS (polybutylene succinate), PBAT (polybutylene co-adipate terephthalate), PVAC (polyvinyl acetate)), a cross-linking agent, and a compatibilizing agent are mixed may be provided. In such a composite resin composition, 28 to 72% by weight of PLA Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, 1 to 3% by weight of compatibilizer may be melt-extruded through an extruder.

The biodegradable composite resin is included in the degradable composite resin in a broad sense, and the degradable composite resin is significantly changed in terms of its chemical structure for a certain period of time under specific environmental conditions according to the American Society for Testing And Materials (ASTM). Therefore, the change of its properties is defined as a plastic that may be measured by standard test methods, and the degradable composite resin is classified into biodegradable, biophotodegradable (composite degradable), and photodegradable plastics. In addition, according to ISO (international Standard Organization), in connection with an ultimate biodegradation, the process wherein the decay of organic matter occurs by the action of microorganisms, and carbon dioxide, water and inorganic salts/biomaterials are finally produced is defined as biodegradation. In the definition of degradable plastic in ISO 472, degradable plastics are classified into biodegradable plastics and degradable plastics.

These biodegradable plastics are plastics which are completely decomposed into water, carbon dioxide, methane gas, biomass, etc. within months to years by simply landfilling plastics used as molded products, packaging materials, hygiene products, agricultural products, etc. without incineration at the time of disposal. In such a composite resin composition, when the amount of the PLA (Poly lactic acid) is less than 28% by weight, it is difficult to obtain the physical properties desired by the user, and when it exceeds 72% by weight, the processability of the composite resin may be deteriorated.

In the composite resin composition, the PLA (Poly lactic acid) is preferably 28%-72% by weight. In addition, when PCL (polycaprolactone) is less than 5% by weight, the tensile strength and yield strength do not reach the level desired by the user, and when it exceeds 20% by weight, mechanical properties may be deteriorated. In the present composite resin composition, the PCL (polycaprolactone) may be preferably 5%-20% by weight.

In addition, in the composite resin composition, when the PBS (polybutylene succinate) is less than 5% by weight, the processability of the obtained resin is significantly deteriorated, and when it exceeds 10% by weight, it does not show any difference as compare with the conventional resin. Meanwhile, the PBS (polybutylene succinate) may be replaced with PBSA (polybutylene succinate adipate). At this time, the PLA (Poly lactic acid) is a stereo complex of PLLA, and PLDA which is an isomer.

In addition, in one embodiment, the PLA (Poly lactic acid) is poly-L-lactide, poly-D-lactide and poly-DL-lactide homopolymer, or PLA may be a copolymer comprising poly-L-lactide, poly-D-lactide and poly-DL-lactide. In this case, in the case of a polymer containing poly-L-lactide, poly-D-lactide, and poly-DL-lactide, 5 to 10% by weight of the poly-D-lactide may be stereo complex. In addition, when the PBAT (polybutylene adipate-co-terephthalate) is less than 5% by weight, mechanical strength and flexibility are deteriorated, and when it exceeds 15% by weight, tearing occurs in the mechanical direction (MD). When the PBAT (polybutylene adipate-co-terephthalate) is mixed in an appropriate ratio, it may have excellent biodegradability and mechanical strength while having improved flexibility.

In the present composite resin composition, the PBAT (polybutylene adipate-co-terephthalate) may be preferably 5% to 15% by weight. In addition, in an embodiment, the PVAC (polyvinyl acetate) may be selected from the group consisting of PVOH (polyvinyl alcohol) linked with a cross-linking agent and derivatives or mixtures thereof. The PVAC (polyvinyl acetate) may be preferably 10 to 20% by weight.

Meanwhile, the compatibilizer is a material for causing strong interfacial adhesion by helping miscibility or compatibility while being present at the interface between constituent components in the composite system. In the present invention, PEG (Poly ethylene glycol), MA (Maleic anhydrate), GAM (Glycidil Maleic anhydrade) may be applied. MAH (Maleic anhydride) may be applied as the compatibilizer.

Hereinafter, the biodegradable composite resin composition according to the present invention will be described in more detail by the following Embodiments and Comparative Examples. However, the present embodiment is only illustratively described to aid understanding the present invention, and is not intended to limit the present invention.

### Embodiment 1

In connection with the biodegradable composite resin composition with enhanced low-temperature processability, 50% by weight of PLA (Poly lactic acid), 10% by weight of PCL (polycaprolactone), 5% by weight of PBS (polybutylene succinate), 5% by weight of PBAT (polybutylene adipate-co-terephthalate), 15% by weight of PVAC (polyvinyl acetate), 3% by weight of a cross-linking agent, and 2% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Embodiment 2

In connection with the biodegradable composite resin composition with enhanced low-temperature processability, 50% by weight of PLA (Poly lactic acid), 15% by weight of PCL (polycaprolactone), 5% by weight of PBS (polybutylene succinate), 15% by weight of PBAT (polybutylene adipate-co-terephthalate), 10% by weight of PVAC (polyvinyl acetate), 4% by weight of a cross-linking agent, and 1% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Embodiment 3

In connection with the biodegradable composite resin composition with enhanced low-temperature processability, 50% by weight of PLA (Poly lactic acid), 20% by weight of PCL (polycaprolactone), 5% by weight of PBS (polybutylene succinate), 10% by weight of PBAT (polybutylene adipate-co-terephthalate), 10% by weight of PVAC (polyvinyl acetate), 2% by weight of a cross-linking agent, and 3% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Embodiment 4

In connection with the biodegradable composite resin composition with enhanced low-temperature processability, 50% by weight of PLA (Poly lactic acid), 15% by weight of PCL (polycaprolactone), 5% by weight of PBS (polybutylene succinate), 5% by weight of PBAT (polybutylene adipate-co-terephthalate), 20% by weight of PVAC (polyvinyl acetate), 4% by weight of a cross-linking agent, and 1% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Embodiment 5

In connection with the biodegradable composite resin composition with enhanced low-temperature processability, 50% by weight of PLA (Poly lactic acid), 5% by weight of PCL (polycaprolactone), 10% by weight of PBS (polybutylene succinate), 15% by weight of PBAT (polybutylene adipate-co-terephthalate), 20% by weight of PVAC (polyvinyl acetate), 4% by weight of a cross-linking agent, and 1% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Comparative Example 1

In connection with the biodegradable composite resin composition with enhanced low-temperature processability, 100% by weight of PLA (Poly lactic acid), 0% by weight of PCL (polycaprolactone), 0% by weight of PBS (polybutylene succinate), 0% by weight of PBAT (polybutylene adipate-co-terephthalate), 0% by weight of PVAC (polyvinyl acetate), 4% by weight of a cross-linking agent, and 0% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Comparative Example 2

In connection with the biodegradable composite resin composition with enhanced low-temperature processability, 850% by weight of PLA (Poly lactic acid), 0% by weight of PCL (polycaprolactone), 5% by weight of PBS (polybutylene succinate), 5% by weight of PBAT (polybutylene adipate-co-terephthalate), 5% by weight of PVAC (polyvinyl acetate), 4% by weight of a cross-linking agent, and 0% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Comparative Example 3

In connection with the biodegradable composite resin composition with enhanced low-temperature processability, 75% by weight of PLA (Poly lactic acid), 5% by weight of PCL (polycaprolactone), 5% by weight of PBS (polybutylene succinate), 5% by weight of PBAT (polybutylene adipate-co-terephthalate), 5% by weight of PVAC (polyvinyl acetate), 4% by weight of a cross-linking agent, and 1% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Comparative Example 4

In connection with the biodegradable composite resin composition with enhanced low-temperature processability, 65% by weight of PLA (Poly lactic acid), 5% by weight of PCL (polycaprolactone), 5% by weight of PBS (polybutylene succinate), 10% by weight of PBAT (polybutylene adipate-co-terephthalate), 12% by weight of PVAC (polyvinyl acetate), 2% by weight of a cross-linking agent, and 2% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

### Comparative Example 5

In connection with the biodegradable composite resin composition with enhanced low-temperature processability, 55% by weight of PLA (Poly lactic acid), 5% by weight of PCL (polycaprolactone), 10% by weight of PBS (polybutylene succinate), 10% by weight of PBAT (polybutylene adipate-co-terephthalate), 15% by weight of PVAC (polyvinyl acetate), 2% by weight of a cross-linking agent, and 3% by weight of a compatibilizer were added and melt-extruded with a twin screw extruder to pelletize. Next, the obtained pellets were molded into 3.2 mm bars at 23° C. according to ASTM D256, and then the notched Izod impact strength of each bar was measured according to ASTM D256, and then shown in Table 2 below.

Table 1 shows the composition ratios of the components constituting the composite resin compositions of Embodiments 1 to 5 and Comparative Examples 1 to 5.

TABLE 1

| | PLA | PCL | PBS | PBAT | | Cross-linking | |
|---|---|---|---|---|---|---|---|
| | (% by | (% by | (% by | (% by | PVAC | agent | Compatibilizer |
| | weight) | weight) | weight) | weight) | (% by weight) | (% by weight) | (% by eight) |
| Embodiment 1 | 50 | 10 | 5 | 15 | 15 | 3 | 2 |
| Embodiment 2 | 50 | 15 | 5 | 15 | 10 | 4 | 1 |
| Embodiment 3 | 50 | 20 | 5 | 10 | 10 | 2 | 3 |
| Embodiment 4 | 50 | 15 | 5 | 5 | 20 | 4 | 1 |
| Embodiment 5 | 50 | 5 | 10 | 15 | 15 | 4 | 1 |
| Comparative | 100 | 0 | 0 | 0 | 0 | 0 | 0 |
| Example 1 | | | | | | | |
| Comparative | 85 | 0 | 5 | 5 | 5 | 0 | 0 |
| Example 2 | | | | | | | |
| Comparative | 75 | 5 | 5 | 5 | 5 | 4 | 1 |
| Example 3 | | | | | | | |
| Comparative | 65 | 5 | 5 | 10 | 12 | 2 | 1 |
| Example 4 | | | | | | | |
| Comparative | 55 | 5 | 10 | 10 | 15 | 2 | 3 |
| Example 5 | | | | | | | |

After measuring the impact strength of the bar processed with the biodegradable composite resin in the form of pellets prepared in Embodiment 1 to 5 and Comparative Example 1 to 5, the results are shown in Table 2.

In addition, the biodegradable composite resin was melt-kneaded at 170-190° C. with a single screw extruder (L/D: 40, diameter 35 mm) to process a 3 mm-thick sheet, and then cut it with a laser cutter according to the splint standard. Thereafter, the temperature was adjusted to 70° C. by using a water bath equipped with an automatic temperature controller, and the cut sheet was immersed for 60 seconds and was molded by using a mockup. The moldability was measured by time and classified into 4 grades as follows to evaluate the low temperature moldability.

TABLE 2

| | Impact strength | |
|---|---|---|
| | (J/m) | Moldability Evaluation |
| Embodiment 1 | 463 | Very excellent |
| Embodiment 2 | 423 | Very excellent |
| Embodiment 3 | 342 | Excellent |
| Embodiment 4 | 368 | Excellent |
| Embodiment 5 | 512 | Good |
| Comparative Example 1 | 63 | Normal |
| Comparative Example 2 | 93 | Normal |
| Comparative Example 3 | 118 | Good |
| Comparative Example 4 | 152 | Good |
| Comparative Example 5 | 321 | Excellent |

In Table 2, the biodegradable composite resins prepared in Embodiment 1 to 5 were found to have superior impact strength as compared with the biodegradable composite resins prepared in the Comparative Examples 1 to 5, and the biodegradable composite resin prepared in Embodiment 1 to 5 was found to have excellent moldability evaluation.

In addition, after processing the sheet, the molding grade of the splint was as follows.

1.—Molding time 30˜60 seconds: very excellent
2.—Molding time 60˜100 seconds: excellent
3.—Molding time 100˜200 seconds: good
4.—Molding time more than 200 seconds: normal

Hereinafter, a method of manufacturing a composite resin using the biodegradable composite resin composition having enhanced low-temperature processability as described above will be described in detail.

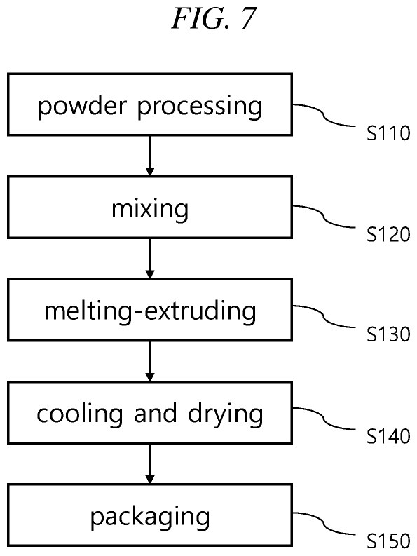

FIG. 7 is a flow chart showing a method for manufacturing a biodegradable composite resin with enhanced low-temperature processability according to an embodiment of the present invention

Referring to FIG. 7, the method for manufacturing a biodegradable composite resin with enhanced low-temperature processability may include a powdery processing step S110, a mixing step S120, a melt-extrusion step S130, a cooling and drying step S140, and a packaging step S150.

In the powdery processing step S110, as the raw materials used in the manufacture of the composite resin, PLA (poly lactic acid), PCL (polycaprolactone), PBS (polybutylene succinate), PBAT (polybutylene adipate-co-terephthalate), PVAC (polyvinyl acetate), cross-linking agents, and compatibilizers may be used. More specifically, 28 to 72% by weight of the PLA (Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, and 1 to 3% by weight of compatibilizer may be mixed.

In the mixing step S120, a double-blade ribbon blender may be used as an equipment for mixing powder raw materials. In the embodiment of the present invention, a double blade ribbon mixer is illustrated as an equipment used for mixing the primary processed product and the remaining raw materials, but any equipment may be applied as long as it is an equipment used for mixing various raw materials according to the embodiments.

In the melt-extrusion step S130, the mixture may be processed by a melt extrusion process using a twin extruder equipped with a raw material supply device. The twin extruder used for melt-extrusion of the mixture is a general known technique used for melt-extrusion of a material, and a detailed description thereof will be omitted

In the cooling and drying step S140, the raw material extruded through the twin extruder equipped with the raw material supply device may be injected into a die, and then the strands extracted through the die may be cooled and dried. At this time, for cooling and drying of the strand, a water cooling type or an air cooling type may be generally applied. Here, the water cooling type requires secondary drying of the PLA composite resin. Accordingly, in an embodiment of the present invention, an cooling and drying method of an air cooling type, which is a type wherein cooling and drying are integrally performed, may be applied.

The strand processed through the extruder has a predetermined temperature. Accordingly, the temperature of the strand may be cooled in the cooling and drying steps. The application of the air cooling system during the cooling and drying steps of the strand is to realize an eco-friendly manufacturing method by excluding the drying step after cooling to reduce power and time. As the air cooling system for cooling the strand, a conventional air cooling system used when cooling an object may be used.

In the embodiment of the present invention, the equipment applied to the sheet cooling is embodied as an air cooling system, but any equipment may be applied as long as it is equipment used to cool the object. In addition, since the water-cooled type must undergo a drying step, the air-cooled type may be applied in the present invention.

In the packaging step S150, the cooled strand is pelletized and then packaged. Here, the specifications of the strand are as follows: density 1.25±0.05 (g/cd), tensile strength 50 (Mpa), tensile activity rate 3.5 to 6 (Gpa), softening temperature 60 to 70° C., shrinkage less than 0.5%, and moisture content less than 200 ppm. In addition, in the step S40 of cooling and drying the melt-extruded strand, the processed strand may be cooled with an air cooling system.

Accordingly, in connection with the biodegradable composite resin with enhanced low-temperature processability, and the composite resin manufactured through the manufacturing method according to an embodiment of the present invention, through the improvement of the main raw material components and the manufacturing method using the same, it is possible to easily post-process products manufactured using the above materials at a low temperature of 60˜70° C., and to meet the biodegradability conditions as a composite resin that is harmless to bio-contact and meets the food contact standard to enable recycling of resources.

Hereinafter, a method for manufacturing a sheet using a biodegradable composite resin having enhanced low-temperature process ability as described above will be described in detail.

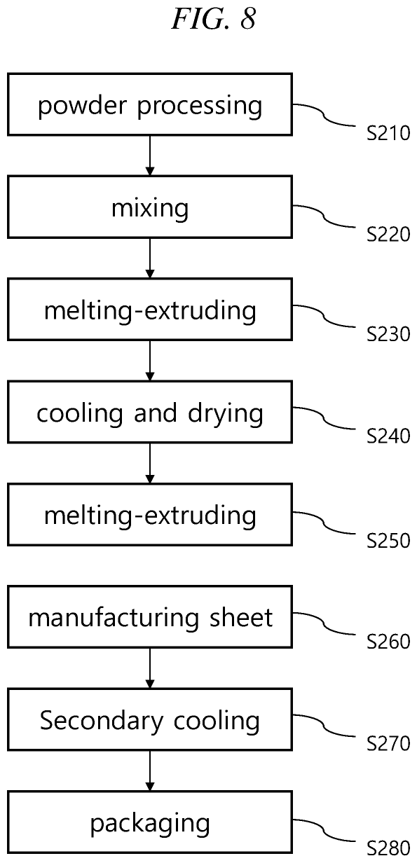

FIG. 8 is a flow chart illustrating a sheet manufacturing method using a biodegradable composite resin with enhanced low-temperature processability according to an embodiment of the present invention.

Referring to FIG. 8, the sheet manufacturing method may comprises; a step S210 for powder processing 28 to 72% by weight of PLA (Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, and 1 to 3% by weight of a compatibilizer; a step S220 for mixing the raw material processed into a powder state with a double blade ribbon blender; a step S230 for melting-extrusion of the mixed raw material using a twin extruder equipped with a raw material supply device; a step S240 for injecting the melt-extruded raw material into a die and then cooling and drying the strands outputted through the die); a step for S250 for melting and extruding the cooled and dried strand using a T-die extruder for sheet manufacturing; a step S260 for manufacturing a sheet by rolling the melt-extruded composite resin through the T-die extruder to control the thickness and to perform a primary cooling; a step for S270 for applying a secondary cooling to the sheet cooled by the primary cooling in a state in which the thickness is adjusted; and a step S280 for cutting and packaging the sheet cooled by the secondary cooling according to standards.

The steps S210 to S240 correspond to steps S110 to S140 of the method for manufacturing a biodegradable composite resin with enhanced low-temperature processability described with reference to FIG. 7, respectively, and as for steps S110 to S140, the description of steps S110 to S140 of FIG. 7 may be referred to.

In the sheet manufacturing and primary cooling step S260, a sheet may be manufactured by rolling the melt-extruded composite resin through the T-die extruder, adjusting the thickness and primary cooling. The extruder used in the sheet manufacturing method according to the embodiment of the present invention may be applied to any extruder as long as it is a general extruder for sheet manufacturing. In adjusting the thickness via rolling and the primary cooling step S260, a 3-axis roller including a cooling roll may be applied. In addition, in sheet manufacturing, if optimal melting temperature control is possible, and as a process guide is generally used, detailed descriptions thereof will be omitted. Here, the specifications of the sheet of the embodiment of the present invention are as follows. Density of 1.25±0.05 (g/cd), Tensile strength of 50 (Mpa), Tensile Activity Rate of 3.5 to 6 (Gpa), Softening Temperature of 60 to 70° C., Shrinkage less than 0.5%, and Moisture content of 200 ppm or less may be set.

In the second cooling step S270, the sheet cooled via a primary cooling step is subject to a secondary cooling while the thickness is adjusted, and in the packaging step S280, the sheet cooled through the secondary cooling may be cut and packaged according to a standard.

According to an embodiment of the present invention, in connection with the biodegradable composite resin with enhanced low-temperature processability, and the composite resin manufactured through the composite resin manufacturing method, the main raw material components and the manufacturing method using the same are eco-friendly improved. In addition, the products using the composite resin prepared in this way may be easily post-processed at a low temperature of 60˜70° C., is harmless to biological contact, conforms to food contact standards, and meets biodegradable conditions to enable recycling of resources.

Although the invention made by the present inventor has been described in detail according to the above embodiments, the invention is not limited to the above embodiments, and may be changed in various ways without departing from the gist of the invention.

What is claimed is:

1. A splint bendable in a curved shape comprising:
a lower splint portion having a plurality of first ventilation holes formed at a certain intervals;
an extension splint portion in which a plurality of extension holes are formed at a certain intervals; and
an upper splint portion in which a plurality of second ventilation holes are formed at a certain intervals, and
where the lower splint portion, the extension splint portion, and the upper splint portion are bent into an arc shape by a first bending, and
wherein the lower splint portion and the upper splint portion are bent at different angles by a second bending of the extension splint portion with a plurality of extension holes.

2. The splint bendable in a curved shape of the claim 1, wherein the extension splint portion comprises a plurality of extension holes and the plurality of extension holes are arranged at a certain interval so that the lower splint portion and the upper splint portion may be bent smoothly by the second bending.

3. The splint bendable in a curved shape of the claim 1, wherein the extension splint portion comprising:
a first extension hole extending which is formed in a predetermined length in a middle of the extension splint portion, and is extended vertically apart by the second bending; and
a second extension hole which is formed on both sides of the first extension hole to have a predetermined length, respectively, and extended vertically apart by the second bending.

4. A splint bendable in a curved shape comprising:
a lower splint having a plurality of first ventilation holes, second ventilation holes, and third ventilation holes formed at a certain interval;
an upper splint portion extending to an upper side of the lower splint portion; and
an extension splint portion extending to one side of the upper splint portion and having a plurality of extension holes formed at a certain interval;
wherein the lower splint portion and the upper splint portion are bent into an arc shape by a first bending, and
wherein the extension splint portion are bent by a second bending to allow a predetermined diameter through plurality of extension holes.

5. A method of forming a splint bendable in a curved shape comprising:
a step for cutting a splint member into a predetermined shape according to a treatment portion applied by the splint;
a step for forming a plurality of extension holes in the extension splint via a perforation process in order to smoothly transform and extend the splint member when the splint member is bent;
a step for forming a plurality of ventilation holes via a perforation process to allow air to communicate with the outside according to the treatment portion;
a first bending step for bending the splint member into a predetermined arc shape so as to surround an outer surface of the treatment portion; and
a second bending step for bending the extension splint portion having the extension hole formed thereon to have a different angle or a predetermined diameter.

6. A biodegradable composite resin composition with enhanced low-temperature processability, A biodegradable composite resin manufactured by mixing PLA (Poly lactic acid), PCL (polycaprolactone), PBS (polybutylene succinate), PBAT (polybutylene adipate-co-terephthalate), PVAC (polyvinyl acetate), a cross-linking agent, and a compatibilizer.

7. The biodegradable composite resin composition with enhanced low-temperature processability of the claim 6, wherein 28 to 72% by weight of PLA (Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4 by weight of a cross-linking agent, 1 to 3% by weight of a compatibilizer are melt and extruded through an extruder.

8. The biodegradable composite resin composition with enhanced low-temperature processability of the claim 7, wherein the PLA is a stereo-complex of PLDA which is an isomer of PLLA.

9. The biodegradable composite resin composition with enhanced low-temperature processability of the claim 7, wherein PLA is a homopolymer such as poly-L-lactide, poly-D-lactide and poly-DL-lactide, or PLA is a copolymer including poly-L-lactide, poly-D-lactide and poly-DL-lactide.

10. The biodegradable composite resin composition with enhanced low-temperature processability of the claim 9, wherein in case of a copolymer including the poly-L-lactide, the poly-D-lactide, and the poly-DL-lactide, 5 to 10% by weight of the poly-D-lactide is a stereo complex.

11. The biodegradable composite resin composition with enhanced low-temperature processability of the claim 7, wherein the PVAC (polyvinyl acetate) is selected from the group consisting of PVOH (polyvinyl alcohol linked with a cross-linking agent and derivatives or mixtures thereof.

12. The biodegradable composite resin composition with enhanced low-temperature processability of the claim 7, wherein the PBS (polybutylene succinate) may be replaced with PBSA (polybutylene succinate adipate).

13. The biodegradable composite resin composition with enhanced low-temperature processability of the claim 7, wherein the compatibilizer is MAH (maleic anhydride).

14. A method of manufacturing a composite resin comprising:
a step S110 for powdery processing 28 to 72% by weight of PLA Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, 1 to 3% by weight of compatibilizer;
a step S120 for mixing the powdery processed raw material by using a double blade ribbon blender;
a step S130 for performing melting-extrusion of the mixed raw material using a twin extruder equipped with a raw material supply device;
a step S140 for injecting the melt-extruded raw material into a die, and then cooling and drying the strands outputted through the die; and
a step S150 for pelletizing the cooled strand through a cutting machine and packaging the pelletized strand.

15. The method of manufacturing a composite resin of the claim 14, wherein the strand has the specification defining density of 1.25±0.05 (g/cm²), tensile strength of 50 (Mpa), tensile activity rate of 3.5˜6 (Gpa), softening temperature of 60˜70° C., shrinkage less than 0.5% and moisture content less than 200 ppm.

16. The method of manufacturing a composite resin of the claim 14, wherein in the step S140 for cooling and drying the melt-extruded strand, the processed strand is be cooled with an air cooling system.

17. A method of manufacturing a sheet by using biodegradable composite resin with enhanced low-temperature processability comprising;
a step S210 for powdery processing 28 to 72% by weight of PLA (Poly lactic acid), 5 to 20% by weight of PCL (polycaprolactone), 5 to 10% by weight of PBS (polybutylene succinate), 5 to 15% by weight of PBAT (polybutylene adipate-co-terephthalate), 10 to 20% by weight of PVAC (polyvinyl acetate), 2 to 4% by weight of cross-linking agent, and 1 to 3% by weight of a compatibilizer;
a step S220 for mixing the raw material processed into a powder state with a double blade ribbon blender;
a step S230 for melting-extrusion of the mixed raw material using a twin extruder equipped with a raw material supply device;
a step S240 for injecting the melt-extruded raw material into a die and then cooling and drying the strands outputted through the die;
a step for S250 for melting and extruding the cooled and dried strand using a T-die extruder for sheet manufacturing;
a step S260 for manufacturing a sheet by rolling the melt-extruded composite resin through the T-die extruder, adjusting a thickness and performing a primary cooling;
a step for S270 for applying a secondary cooling to the sheet cooled by the primary cooling in a state in which the thickness is adjusted; and
a step S280 for cutting and packaging the sheet cooled by the secondary cooling according to standards.

18. The method of manufacturing a sheet by using biodegradable composite resin with enhanced low-temperature processability of the claim 17, wherein during the adjustment of a thickness via a rolling and the primary cooling step S260, a three-axis roller including a cooling roll is applied.
